(19) United States
(12) Patent Application Publication
(10) Pub. No.: US 2019/0231512 A1
(43) Pub. Date: Aug. 1, 2019

(54) ULTRA-LOW PROFILE WOVEN, KNITTED, AND BRAIDED TEXTILES AND TEXTILE COMPOSITES MADE WITH HIGH TENACITY YARN

(71) Applicant: THE SECANT GROUP, LLC, Telford, PA (US)

(72) Inventors: Amanda WEBER, Macungie, PA (US); Mevlut TASCAN, Breinigsville, PA (US); Carissa SMOOT, Harleysville, PA (US); Andrew METZGER, Lafayette Hill, PA (US)

(21) Appl. No.: 16/264,216

(22) Filed: Jan. 31, 2019

Related U.S. Application Data
(60) Provisional application No. 62/624,546, filed on Jan. 31, 2018.

(51) Int. Cl. A61F 2/07; D04B 1/16; D03D 1/00; D04B 21/00; D03D 13/00; D03D 15/00; D06M 15/507; D06M 15/564

(52) U.S. Cl. CPC A61F 2/07; D04B 1/16; D03D 1/00; D04B 21/00; D03D 13/004; D06M 2101/32; D06M 15/507; D06M 15/564; D10B 2509/06; D03D 2700/0174; D10B 2509/08; D03D 15/00

(57) ABSTRACT

Textiles for endovascular and other medical applications having a low-profile are provided. The textiles are woven, knit or braided and are formed of yarns having low (<17) denier and high tenacity (at least 7 grams per denier). The resulting textiles have a low profile, high suture retention and extremely low water permeability.

### RELATED APPLICATIONS

This application claims the benefit of, and priority to, U.S. App. No. 62/624,546 filed Jan. 31, 2018, which is hereby incorporated by reference in its entirety.

### FIELD OF THE INVENTION

The present invention is directed to ultra-low profile woven, knitted, and braided textiles and textile composites made with high tenacity (HT) yarn.

### BACKGROUND OF THE INVENTION

In vascular stent grafts for endovascular aneurysm repair (EVAR) procedures, the textile occupies up 30% of the delivery device's real estate. The current state of the art in woven EVAR and transcatheter valve replacement/repair (TVR) graft material is a thickness of≥61 micrometers (μm). This thickness limits the size of the delivery catheter to 14 fr, which excludes many patients with smaller femoral arteries, especially in women.

### BRIEF DESCRIPTION OF THE INVENTION

Exemplary embodiments seek to overcome this and other limitations in textiles for medical and other applications by providing a lower profile textile for use, for example, in woven endovascular stent grafts and transcatheter heart valve skirt and cuff fabrics that are <60 μm in bare textile thickness but which still exhibits the strength and permeability characteristics desirable for the intended application. Additionally, providing exemplary embodiments having reduced textile profile allows delivery of these devices to patients through smaller delivery systems, expanding the potential servable patient population. Additionally, exemplary embodiments also achieve knit textiles that have a thickness significantly less than current knit textiles used in medical applications.

In an embodiment, an endovascular textile includes a yarn having a denier of less than 17 denier and a tenacity of at least 7 grams per denier. The textile may be of woven, braided, or knit construction. The thickness of the bare textile is less than 100 micrometers, and less than 60 micrometers when the textile is woven, with coated woven endovascular textiles having a thickness less than 70 micrometers.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 is a schematic representation of a region of a plain weave implantable textile in accordance with an exemplary embodiment.

FIG. 2 is a schematic representation of a region of a weft rib weave implantable textile in accordance with an exemplary embodiment.

FIG. 3 is a schematic representation of a region of a 2×2 twill weave implantable textile in accordance with an exemplary embodiment.

FIG. 4 is a coated single bar (1-0/1-2) composite implantable knit textile, according to an embodiment.

FIG. 5 is a coated 2GB (1-0/1-2) composite implantable knit textile, according to an embodiment.

FIG. 6 is an uncoated plain weave implantable textile, according to an embodiment.

FIG. 7 is a PGS coated plain weave composite implantable textile, according to an embodiment.

FIG. 8 is an uncoated weft rib weave implantable textile, according to an embodiment.

FIG. 9 is an exemplary graft structure, according to an embodiment.

FIG. 10 is an exemplary heart valve, according to an embodiment.

### DETAILED DESCRIPTION OF THE INVENTION

Provided is a low profile implantable textile for endovascular aneurysm repair (EVAR) and transcatheter aortic valve replacement (TAVR) procedures.

Embodiments of the present disclosure, for example, in comparison to concepts failing to include one or more of the features disclosed herein, provide an endovascular textile comprising a high tenacity yarn, at least 7 grams per denier, of less than 17 denier (dn), and having a textile thickness of less than 200 micrometers.

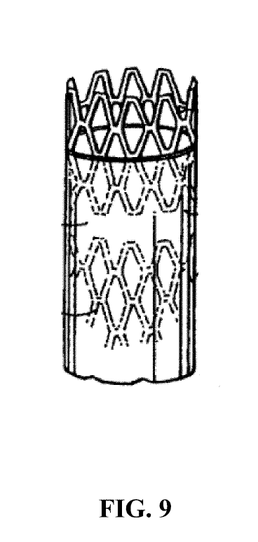

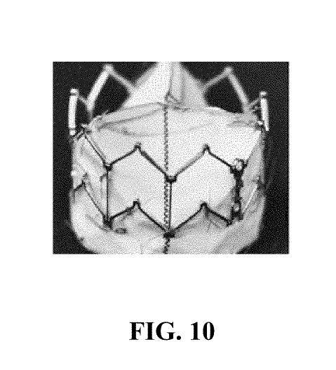

During transcatheter procedures, such as endovascular aneurysm repair (EVAR) and transcatheter aortic valve replacement (TAVR) procedures, an endovascular textile may be inserted into the patient to reinforce the affected region of the blood vessel. The regions of the body for which such inserts may be used are limited by the diameter of the affected blood vessels. Low profile textiles offer the opportunity to apply life saving surgical techniques to regions of the body in which the blood vessels are of smaller diameter. Additionally, by reducing the profile of the endovascular implant, the implant takes up less space within the device's delivery catheter, further enhancing delivery to additional regions of the body. FIGS. 9 and 10 schematically illustrate a basic form of graft and valve structures, respectively, for use with textiles in accordance with exemplary embodiments, although it will be appreciated that specific conformations vary widely.

In order to create a thinner fabric without sacrificing the functional properties required in a vascular implant, a low denier (<17 denier), high tenacity (>7 grams/denier) yarn is used to allow higher yarn densities. This results in the ability to achieve greater suture retention and strength properties in the fabric, while also decreasing the thickness of the textile construction. In some embodiments, the low denier, high tenacity yarn is a multi-filament yarn.

By utilizing a fine denier yarn of less than 17 denier, with high tenacity characteristics in conjunction with a stable construction such as weft rib weave, exemplary embodiments can maintain a similar suture retention strength and water permeability as thicker conventional textiles, while still also lowering its profile. The textile may be formed from various woven, knit, or braided constructions, including but not limited to a double needle bar knit, tricot warp knit, a plain weave, twill weave, rib weave (e.g., warp rib or weft rib), satin weave, sateen weave, mock leno weave, and/or herringbone weave. In some embodiments, the textile is formed from a plain weave, a 2x2 twill weave, weft rib, or satin weave. In some embodiments, the textile is formed from a double needle bar knit, or tricot warp knit. In some embodiments, the denier of the HT yarn is 10 denier or greater, 12 denier or greater, 14 denier or greater, greater than 15 denier, about 16 denier, less than 17 denier and combinations thereof.

The yarn can be formed of any suitable material of construction that can be suitably used for endovascular textiles, including, for example, poly(ethylene terephthalate) (PET), polytetrafluoroethylene (PTFE), and collagen. In certain presently preferred embodiments, the yarn includes PET. In some embodiments, the yarn has a substantially round cross-section.

In some embodiments, the textile is woven. In an exemplary embodiment, a method of making the woven endovascular textile includes weaving high tenacity PET yarn with a denier less than 17 and tenacity greater than 7 grams per denier. In one embodiment, the textile is woven using a 16 denier high tenacity PET yarn having a 10z twist (i.e., 10 turns per inch) for the warp and a 16 denier high tenacity PET yarn having zero twist for the weft. If a twist is imparted, after the initial twisting process, the yarn may be autoclaved. This aids in thermally setting the twist in the yarn to add dimensional stability before beginning the weaving process.

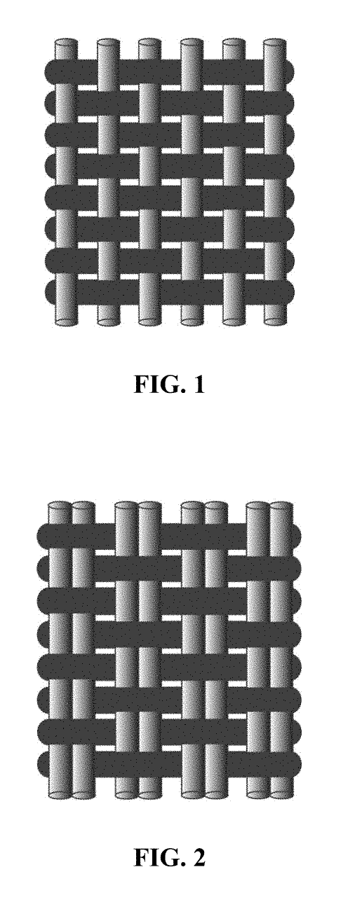

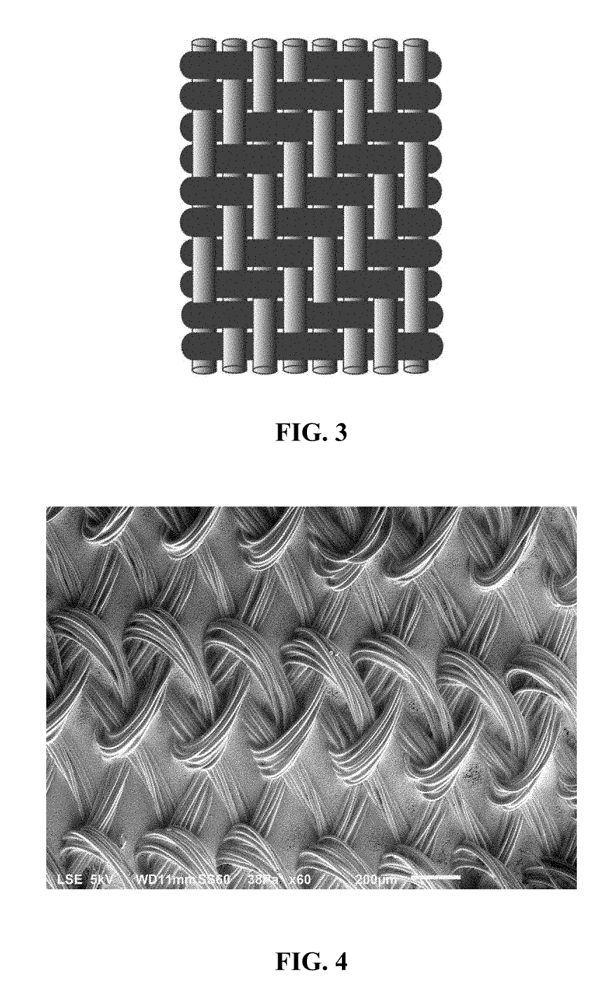

Among the various weave patterns that may be employed, are a plain weave, a twill weave, a warp rib, and a weft rib. Illustrations of exemplary embodiments employing each of a plain weave, weft rib weave, and 2×2 twill weave are shown in FIGS. 1, 2, and 3 respectively.

Textiles in accordance with exemplary embodiments have an end count preferably less than 450 ends per inch (EPI) and with less than 170 picks per inch (PPI). In some embodiments, the textile is a woven textile having between 120-320 EPI and 80-148 PPI on loom with a weft rib structure. Optionally, several warp ends may be bundled and woven as one to reinforce each end. This may result in textile structures having improved suture retention strength characteristics.

After weaving, the resulting textile may be scoured to remove any lubrications or stains on the fabric and then heat set, such as, for example, on a stainless-steel drum that is placed in an atmospheric oven or fed continuously through a heated tenter frame. Once the fabric is dry, it may be optionally calendared or heat pressed to compress the fabric resulting in an even thinner profile.

In one embodiment, the fabric is cleaned and then heat set at about 205° C. (about 400° F.) for dimensional stability. In one embodiment, the fabric is calendared at about 150° C. (about 300° F.) using a cotton wrapped roller and stainless-steel roller.

The resulting bare textile has a thickness that generally ranges between 35 and 60 or 70 micrometers. In some embodiments, the textile thickness may be greater than 35 micrometers, greater than 40 micrometers, and greater than 50 micrometers and is generally less than 70 micrometers, such as less than 60 micrometers, and any range or subrange therebetween.

In other embodiments, a textile may be formed by low denier, high tenacity yarns by braiding or knitting. Textiles knit in accordance with exemplary embodiments are produced using a single continuous low denier, high tenacity yarn and may be knit using any suitable knitting technique, including both those accomplished using either single or double guide bar (GB) techniques. The resulting bare knit textiles have a thickness less than 140 micrometers, such as about 120 micrometers or less, such as about 110 micrometers. As with woven textiles in accordance with exemplary embodiments, it will be appreciated that the thickness of the textile may be further reduced by introducing heat pressing after knitting to compress its thickness and increase its density and may also be calendared.

It will be appreciated that in various medical applications, such as heart valve replacement or repair, it may be desirable to have a water impermeable barrier. Thus the bare textile may be coated after weaving with a bioresorbable or non-bioresorbable materials to reduce water permeability, thereby forming a composite textile.

Suitable non-bioresorbable coating materials include polyurethanes (PU) and various elastomers. Suitable resorbable materials include, but are not limited to, polycaprolactone (PCL), polylactic acid (PLA), polyglycolic acid (PGA), poly(lactic-co-glycolic acid) (PLGA), poly(glycerol sebacate) (PGS), lysine-poly(glycerol sebacate) (KPGS), poly(glycerol sebacate urethane) (PGSU), amino-acid incorporated PGS, and combinations thereof. In some embodiments, the coating may be applied by spray coating, dip coating, or lamination techniques.

In some embodiments, the water permeability of the textile is less than 500 mL/min/cm², less than 400 mL/min/cm², less than 375 mL/min/cm², less than 350 mL/min/cm², less than 325 mL/min/cm², less than 300 mL/min/cm², less than 275 mL/min/cm², less than 250 mL/min/cm², less than 225 mL/min/cm², less than 200 mL/min/cm², less than 150 mL/min/cm², less than 100 mL/min/cm², less than 75 mL/min/cm², less than 50 mL/min/cm², less than 30 mL/min/cm², less than 20 mL/min/cm², less than 10 mL/min/cm², less than 5 mL/min/cm², less than 3 mL/min/cm², and/or less than 1 mL/min/cm².

It will further be appreciated that the application of a coating to form a composite textile increases the thickness of the textile. In some embodiments, the coated woven textile composite thickness is less than 110 micrometers, such less than 90 micrometers, preferably less than 70 micrometers, and may be greater than 40 micrometers, greater than 50 micrometers, and greater than 60 micrometers, for example, or any range or subrange of any of the foregoing.

In exemplary embodiments, when coating a low profile knit structure, an elastomeric polymer may be used that elongates with the textile and maintains its low water permeability characteristic. The elastomeric coating maintains its integrity while conforming to unique geometries and, in combination with the stretchiness of the knit structure, is able to conform with the body's internal movements and pulsation. In some embodiments, the coated knit textile composite thickness is preferably less than 170 micrometers, such as less than 150 micrometers, less than 130 micrometers or less than 120 micrometers, but in some embodiments is still greater than 70 micrometers, greater than 80 micrometers, greater than 90 micrometers, or any range or subrange of any of the foregoing.

The composite textiles may thereafter be calendared to further decrease the thickness.

### EXAMPLES

The invention has been reduced to practice and exemplary embodiments have been formed in which plain and weft rib woven textiles were formed using less than 17 denier HT polyester (PET) having a tenacity of greater than 7 grams per denier.

### Example 1

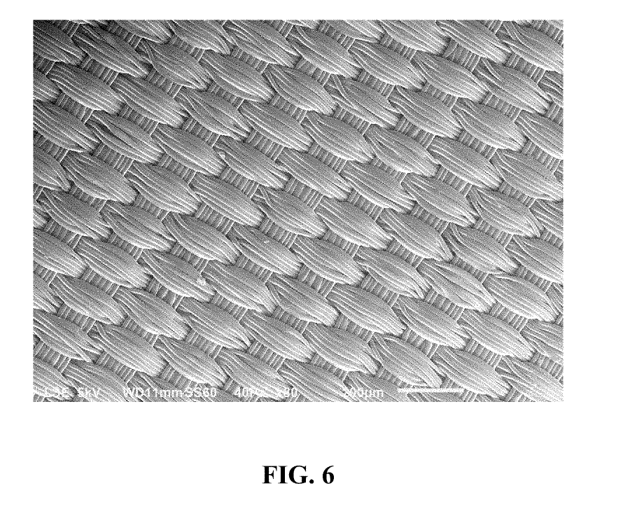

A plain weave textile was woven with 16/10z HT PET warp yarns and weft yarns were 16 denier HT PET yarns with zero twist. The textile of Example 1 is shown in FIG. 6.

### Example 2

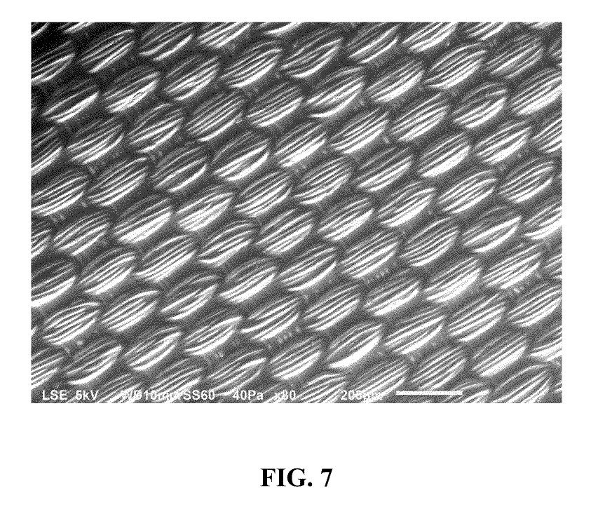

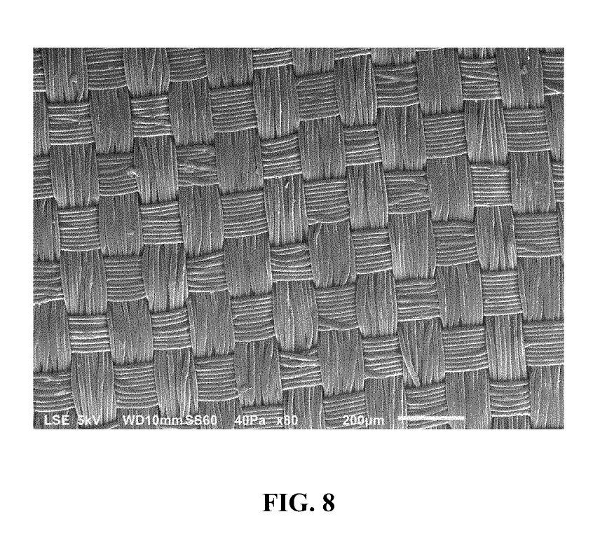

The plain weave textile of Example 1 was subsequently coated with a resorbable coating of poly(glycerol sebacate) (PGS). The textile of Example 2 is shown in FIG. 7.

### Example 3

The warp and weft yarns used in Example 1 were used to weave a weft rib textile.

The Examples 1 through 3 were measured for suture retention and water permeability and compared against a conventional plain weave PET graft textile woven from 20 denier PET multifilament yarn having a tenacity less than 7 grams per denier and the results are shown in Table 1 below.

TABLE 1

| | Weave | | | | Suture | Water |
|---|---|---|---|---|---|---|
| | Struc- | | | Thickness | Reten- | Permeability |
| Example | ture | EPI | PPI | (microns) | tion (N) | (mL/min/cm²) |
| Compar- | Plain | 300 | 150 | 61 | 8.1 | 141 |
| ative | | | | | | |
| 1 | Plain | 364 | 142 | 58.7 | 12.15 | 129 |
| 2 | Plain | 364 | 142 | 64.6 | not | <1 |
| | | | | | tested | |
| 3 | Weft | 342 | 139 | 58.5 | 14.1 | 208 |
| | Rib | | | | | |

The 16 denier HT PET out-performed the conventional 20 denier PET by having improved suture retention in combination with reduced thickness. The resulting composite textile exhibited a composite thickness still less than 70 micrometers and a water permeability of less than 1 mL/min/cm².

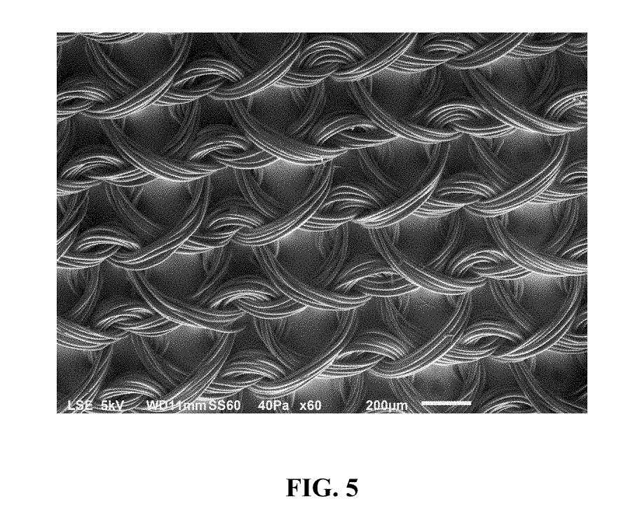

Knit structures have also been reduced to practice. A single bar (1-0/1-2) knit textile coated with PGS to form a composite knit textile is shown in FIG. 4, which has a thickness of about 124 micrometers, in which the bare knit textile had a thickness of about 111 micrometers. A coated 2GB (1-0/1-2) knit textile coated with PGS to form a composite knit textile is shown in FIG. 5. The double bar composite knit textile of FIG. 5 has a thickness of about 168 micrometers, in which the bare knit textile had a thickness of about 164 micrometers. It will be appreciated that a single bar textile will generally have a thinner profile than a double guide bar pattern when using the same yarn, due to the increased amount of that yarn needed to create the structure.

The textiles produced by the materials and techniques described herein are described primarily for use in endovascular applications, such as straight or bifurcated woven endovascular grafts; endovascular aneurysm repair (EVAR) and transcatheter aortic valve replacement (TAVR) procedures; and knit endovascular grafts, but may also be used in various other applications including hernia repair, urology, incontinence, and breast augmentation; coated braided sutures and tethers, all by way of example.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. An implantable medical textile comprising:
a yarn having a denier of less than 17 denier and a tenacity of at least 7 grams per denier;
wherein the textile is woven, braided, or knit; and
wherein the textile is less than 200 micrometers thick.

2. The textile of claim 1, wherein the textile is woven and the textile is less than 60 micrometers thick.

3. The textile of claim 2, wherein the textile includes a plain weave, a twill weave, a warp rib weave, or a weft rib weave.

4. The textile of claim 3, wherein the textile is a weft rib weave.

5. The textile of claim 1, wherein the textile is knit and the textile is less than 140 micrometers thick.

6. The textile of claim 5, wherein the textile is warp knit.

7. The textile of claim 1, wherein the yarn denier is between 15 denier and 17 denier.

8. The textile of claim 1, wherein the yarn denier is about 16 denier.

9. The textile of claim 1, wherein the textile is woven, with the textile having 16 denier polyethylene yarn with about 10 twists per inch in the warp direction and 16 denier polyethylene yarn in the weft direction.

10. The textile of claim 1, wherein the textile exhibits a water permeability of less than 500 milliliters/minute/centimeter².

11. The textile of claim 1, wherein the textile exhibits a water permeability of less than 100 milliliters/minute/centimeter².

12. The textile of claim 1, further comprising a bioresorbable coating overlying the textile, the coating selected from the group consisting of polycaprolactone (PCL), polylactic acid (PLA), polyglycolic acid (PGA), poly(lactic-co-glycolic acid) (PLGA), poly(glycerol sebacate) (PGS), Lysine-poly(glycerol sebacate) (KPGS), poly(glycerol sebacate urethane) (PGSU), amino-acid incorporated PGS, and combinations thereof, thereby forming a composite textile.

13. The textile of claim 12, wherein the textile is woven, and the composite textile exhibits a water permeability of less than 5 milliliters/minute/centimeter² and has a thickness of between 50 micrometers and 75 micrometers.

14. The textile of claim 12, wherein the coating is applied to the textile after weaving or knitting.

15. The textile of claim 12, wherein the textile is knit, and the composite textile exhibits a water permeability of less than 5 milliliters/minute/centimeter² and has a thickness of between 100 micrometers and 200 micrometers.

16. The textile of claim 1, further comprising a polyurethane coating.

17. An implantable medical textile comprising:
a woven textile formed from yarn in each of the warp and weft having a denier of at least 10 denier and less than 17 denier and the yarn further having a tenacity of at least 7 grams per denier,
wherein the textile is less than 60 micrometers thick and has a water permeability of less than 200 milliliters/minute/centimeter².

18. The medical textile of claim 17, further comprising a coating overlying the textile to form a composite textile, the composite textile being less than 70 micrometers thick and having a water permeability of less than 5 milliliters/minute/centimeter².
